(19) United States
(12) Patent Application Publication
(10) Pub. No.: US 2017/0071842 A1
(43) Pub. Date: Mar. 16, 2017

(54) CLEANSING AGENTS CONTAINING BIOSURFACTANTS AND HAVING PREBIOTIC ACTIVITY

(71) Applicant: Henkel AG & Co. KGaA, Duesseldorf (DE)

(72) Inventors: Heike Schelges, Willich (DE); Maria Tretyakova, Dortmund (DE); Brigitte Ludwig, Krefeld (DE)

(73) Assignee: Henkel AG & Co. KGaA, Duesseldorf (DE)

(21) Appl. No.: 15/254,554

(22) Filed: Sep. 1, 2016

(30) Foreign Application Priority Data
Sep. 14, 2015 (DE) 10 2015 217 507.3

(51) Int. Cl. A61K 8/60; A61K 8/46; A61K 9/00; A61Q 19/10; A61K 31/7028

(52) U.S. Cl. CPC A61K 8/602; A61Q 19/10; A61K 31/7028; A61K 9/0014; A61K 8/463; A61K 2800/70; A61K 2800/596

(57) ABSTRACT

Cosmetic cleansing agents include biosurfactants and have prebiotic activity. The prebiotic effect is produced by combining biosurfactants with anionic surfactants. The cosmetic cleansing agent according to the invention also has excellent foaming properties and washing properties. The present invention also relates to the use of biosurfactants as a prebiotically effective component of cosmetic cleansing agents.

### FIELD OF THE INVENTION

The present invention generally relates to cosmetic cleansing agents that include biosurfactants and have prebiotic activity.

### BACKGROUND OF THE INVENTION

Inflammatory skin conditions are caused by harmful bacteria, for example Propionibacterium acnes, that are always present on the skin, but proliferate more rapidly under specific conditions and thereby result in “unclear skin” or acne, for example. In this respect, said harmful bacteria are essentially microorganisms (bacteria and fungi) which are classified as pathogenic.

On this point, the resident bacterial skin flora also includes other bacterial species which are not only harmless, but the growth of which also keeps the harmful bacteria at bay, and therefore said bacterial species have a significant protective function. These bacterial species include first and foremost coagulase negative staphylococci, for example S. epidermidis.

Nonselective antibacterial active ingredients, as are used in commercially available cosmetics for preventing and fighting acne for example, kill not only the undesired skin bacteria but also desired skin bacteria and thereby cause the biological balance to be disturbed, and this can have various undesired consequences. Some other ingredients in cosmetic cleansing agents, for example surfactant mixtures, may potentially contribute to disturbance of this type.

It is therefore necessary to have means available that selectively promote, at the application site on the skin, the growth and/or the survivability of the desirable bacteria in the skin flora over the growth and/or the survivability of the undesired bacteria in the skin flora. Substances of this type are also referred to as being “prebiotic”.

DE 10 2004 011968 A1 discloses plant extracts, in particular coniferous wood extracts, which have a prebiotic effect on the skin.

Furthermore, it is becoming ever more important for cosmetic ingredients to be sustainable and this is increasingly demanded by consumers and manufacturers of cosmetic cleansing agents.

Biosurfactants are surface-active substances of microbial origin that can be produced using plant-oil or sugar substrates. Some of these substrates can consist of agricultural waste such as rice husks or wastewater from the sugar industry, and so in this case no raw materials for food production are wasted. Biosurfactants thus satisfy the requirements of sustainability since they are produced from renewable resources. Biosurfactants are used in domestic cleaning agents, washing detergents and dishwasher detergents (e.g. U.S. Pat. No. 5,520,839, DE 19600743 A1), as well as in various cosmetic cleansing agents (e.g. WO 2014/095367 A1, WO 2013/098066 A2).

However, biosurfactants generally have poorer foaming behavior than anionic surfactants for example. WO 2013/098066 A2 discloses surfactants that can be used in cosmetic cleansing agents in combination with other surfactants and fatty acids. However, fatty acids often impair the foaming behavior.

It is therefore desirable to provide cosmetic cleansing agents that have a prebiotic effect, combined with good washing properties, including good foaming behavior.

Furthermore, other desirable features and characteristics of the present invention will become apparent from the subsequent detailed description of the invention and the appended claims, taken in conjunction with this background of the invention.

### BRIEF SUMMARY OF THE INVENTION

A cosmetic cleansing agent having prebiotic effectiveness, characterized in that it includes one or more biosurfactants in combination with one or more anionic surfactants.

The use of biosurfactants as a prebiotically effective component of cosmetic cleansing agents having a prebiotic effect on skin.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

It was surprising to find that cosmetic cleansing agents that include biosurfactants as the surfactants in combination with an anionic surfactant are prebiotically effective. The ingredients included in the cosmetic cleansing agents according to the invention are capable of largely meeting the requirements in terms of sustainability and biodegradability.

The present invention thus relates to:

1. A cosmetic cleansing agent having prebiotic effectiveness,
characterized in that it includes one or more biosurfactants in combination with one or more anionic surfactants.
2. The cosmetic cleansing agent according to point 1, which includes, based in each case on the total weight of the cosmetic cleansing agent:
(a) 1 to 20% by weight of a biosurfactant/biosurfactants and
(b) 1 to 10% by weight of one or more anionic surfactants.
3. The cosmetic cleansing agent according to point 1 or 2, which includes
1 to 10% by weight of the surfactant (a) and
3 to 10% by weight of the anionic surfactant (b).
4. The cosmetic cleansing agent according to one of the preceding points, which includes a glycolipid, a lipopeptide or a combination thereof as the biosurfactant (a).
5. The cosmetic cleansing agent according to one of the preceding points, wherein the biosurfactant (a) is selected from rhamnolipids, sophorolipids, mannosylerythritol lipids, surfactins, fatty acyl glutamates, fatty acyl glycinates and combinations thereof
6. The cosmetic cleansing agent according to point 5, wherein the sophorolipid is a mixture of the acidic form and the lactone form, wherein 20 to 60% by weight is in the acidic form.
7. The cosmetic cleansing agent according to point 5, wherein the rhamnolipid is a mixture of a mono- and dirhamnolipid, which are each derived from 3-hydroxydodecanoic acid and/or 3-hydroxyundecanoic acid.
8. The cosmetic cleansing agent according to one of the preceding points, wherein the anionic surfactant (b) is selected from linear alkyl sulfates having 8 to 24 C atoms, ethylene oxide addition products thereof, and combinations thereof
9. The cosmetic cleansing agent according to one of the preceding points, wherein the anionic surfactant (b) is an alkyl ether sulfate having 10 to 18 C atoms, preferably 12 to 14 C atoms, and 1 to 6 ethylene oxide units, preferably a lauryl ether sulfate having 2 to 4 ethylene oxide units.
10. The cosmetic cleansing agent according to one of the preceding points, which also includes one or more cosmetically acceptable preservatives in a total amount of 0.05 to 1% by weight or 0.1 to 1% by weight or 0.2 to 0.7% by weight, based on the total weight of the cosmetic cleansing agent.
11. The cosmetic cleansing agent according to one of the preceding points, wherein the cosmetic cleansing agent includes 0.5% by weight or less of free fatty acid, and preferably no free fatty acid.
12. The cosmetic cleansing agent according to one of the preceding points, which is formulated as a body cleansing agent, face cleansing agent or agent for cosmetically treating acne.
13. The cosmetic use of a cleansing agent according to one of the preceding claims for treating acne.
14. The use of biosurfactants as a prebiotically effective component of a cosmetic cleansing agent having a prebiotic effect on the skin.
15. The use of biosurfactants in combination with anionic surfactants as a prebiotically effective component of cosmetic cleansing agents having a prebiotic effect on the skin.

According to the invention, “prebiotic effect” is understood to mean that the growth and/or the survivability of the desired, in particular skin-friendly, skin bacteria or microflora is promoted over the growth and/or the survivability of the undesired, in particular skin-unfriendly, skin bacteria or microflora. This is achieved according to the invention in particular in that the cosmetic agent, which includes a biosurfactant and an anionic surfactant, inhibits the growth of the undesired skin bacteria and does not directly influence the growth of the desired skin bacteria or even increases the growth of the desired skin bacteria.

According to the invention, the term “skin” is preferably understood to mean skin itself, in particular human skin, but also the mucus membrane and skin appendages, provided that they include living cells, in particular a hair follicle, hair root, hair bulb, the ventral epithelium of the nail bed (lectulus), and sebaceous glands and sweat glands.

The desired skin bacteria are preferably benign and/or non-pathogenic and/or skin-friendly skin bacteria and/or coagulase negative staphylococci, in particular S. epidermis, S. hominis, S. warneri, S. saprophyticus, S. xylosus, S. capitis or S. simulans, particularly preferably S. epidermidis and/or S. warneri.

The undesired skin bacteria, the growth of which is inhibited, are in particular Propionibacterium acnes.

Preferably, the cosmetic agent according to the invention, which is prebiotically effective on skin, is suitable for restoring or stabilizing the naturally occurring healthy microbial balance in the skin flora.

The present invention also relates to the use of biosurfactants, preferably in combination with anionic surfactants, as a prebiotically effective component of cosmetic cleansing agents having a prebiotic effect on skin. The use seeks in particular to inhibit the growth of the undesired skin bacteria, and not to affect the growth of the desired skin bacteria or to even promote said growth of the desired skin bacteria.

In terms of preferred embodiments and ingredients, the same applies, mutatis mutandis, to preferred uses as to the cosmetic agent according to the invention.

The present invention relates to the cosmetic, non-therapeutic use of cleansing agents according to the invention for treating acne.

The cosmetic cleansing agent according to the invention includes, as an essential component thereof, one or more biosurfactants.

Biosurfactants are understood to be substances that are formed by microorganisms and are often expelled from the cell. Like conventional surfactants, biosurfactants are surface-active substances that reduce the surface tension of liquids and thereby promote the mixing of aqueous (hydrophilic) and water-repellent (hydrophobic) phases. Biosurfactants can be produced under gentle production conditions that require little energy. They are generally highly biodegradable and are very environmentally friendly. Moreover, they are non-toxic, nor are any toxic byproducts produced during the production thereof. Carbohydrates, in particular sugar, e.g. glucose, and/or lipophilic carbon sources such as fats, oils, partial glycerides, fatty acids, fatty alcohols, long-chain saturated or unsaturated hydrocarbons, are used as raw materials for the microbial production of the biosurfactants. According to the invention, the biosurfactants are preferably biosurfactants produced by fermentation.

Biosurfactants include glycolipids, lipopeptides, lipoproteins, fatty acids, phospholipids, neutral lipids and polymeric surfactants (e.g. emulsan), which can all also be used in the present invention.

Glycolipids that can be used in the present invention are compounds in which one or more monosaccharide units are glycosidically bonded to a lipid moiety. Examples of glycolipids as biosurfactants that can be used according to the invention are rhamnolipids, sophorolipids, mannosylerythritol lipids and trehalose lipids. Among these, rhamnolipids, sophorolipids, mannosylerythritol lipids and combinations thereof are preferred.

Rhamnolipids are obtained from bacteria of the genus Pseudomonas, in particular from Pseudomonas aeruginosa, preferably when grown on hydrophobic substrates such as n-alkanes or plant oils. Other glycolipids, for example glucose lipids, cellobiose lipids or trehalose lipids, are produced by other microorganisms on different substrates. According to the invention, mannosylerythritol lipids are also preferred glycolipid biosurfactants; they are produced by Pseudozyma sp., Candida antarctica and Ustilago sp. bacteria.

According to the invention, rhamnolipids have the following general formula:

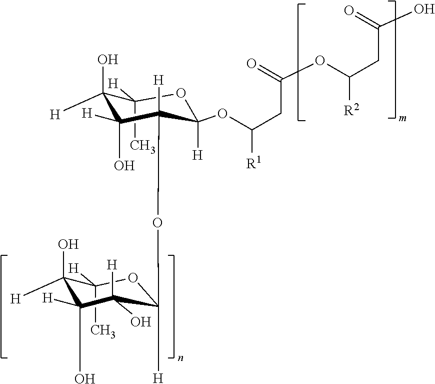

where m is 2, 1 or 0,
n is 1 or 0,
R¹ and R² are, independently of one another, the same or a different organic functional group having 2 to 24, preferably 5 to 13 carbon atoms, in particular a substituted or unsubstituted, branched or unbranched alkyl functional group, which can also be unsaturated, the alkyl functional group preferably being a linear saturated alkyl functional group having 8 to 12 carbon atoms, more preferably a nonyl or a decyl functional group or a mixture thereof.
Salts of these compounds are also included according to the invention.

In the present invention, the term “dirhamnolipid” is understood to mean compounds of the above formula or the salts thereof in which n is 1.

Accordingly, “monorhamnolipid” is understood in the present invention to mean compounds of the general formula or the salts thereof in which n is 0.

Mixtures of mono- and dirhamnolipids can preferably be used according to the invention. In this case, the ratio of monorhamnolipid to dirhamnolipid is preferably approximately 2:1 to 4:1, more preferably 2.5:1 to 3:1. Particularly preferred are those mixtures of mono- and dirhamnolipids in which, in the above formula, R¹ and R², independently of one another, represent a linear nonyl or decyl functional group. In the latter case, these are rhamnolipids that are each derived from 3-hydroxydodecanoic acid and/or 3-hydroxyundecanoic acid. Mixtures of this type can for example be obtained commercially under the name Rhamnolipid R90, R95 or R98 from Agae Technologies, USA, the number indicating the purity in each case. Rhamnolipid R90 can be used particularly preferably according to the invention.

Sophorolipids are produced by fermentation using yeasts such as Candida bombicola (also known as Torulopsis bombicola), Yarrowia lipolytica, Candida apicola (Torulopsis apicola) and Candida bogoriensis, by growing said yeasts on sugars, hydrocarbons, plant oils or mixtures thereof

Sophorolipids have the following formulae (1) (lactone form) and (2) (free acid), the two forms typically being provided in a mixture.

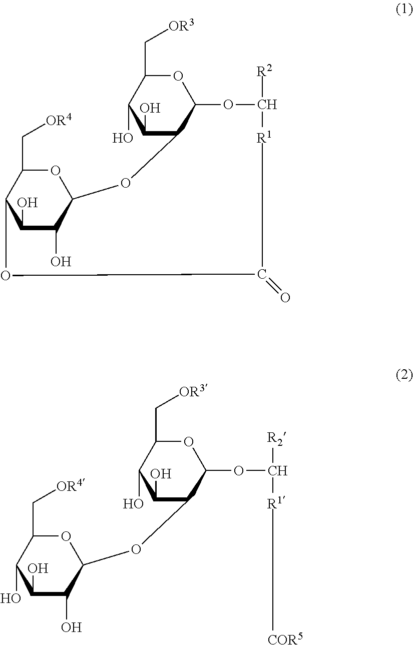

where R¹ and R¹′ represent, independently of one another, saturated hydrocarbon chains or a monounsaturated or polyunsaturated, in particular monounsaturated, hydrocarbon chain having 8 to 20, in particular 12 to 18, hydrocarbon atoms, more preferably 14 to 18 hydrocarbon atoms, which can be linear or branched and can include one or more hydroxy groups,
R² and R²′ represent, independently of one another, a hydrogen atom or a saturated alkyl functional group or a monounsaturated or polyunsaturated, in particular monounsaturated, alkyl functional group having 1 to 9 carbon atoms, preferably 1 to 4 carbon atoms, which can be linear or branched and can include one or more hydroxy groups, and
R³, R³′, R⁴ and R⁴′ represent, independently of one another, a hydrogen atom or an acetyl group.

Sophorolipids in which R¹ and R¹′ are monounsaturated, linear hydrocarbon chains having 15 carbon atoms are preferred. It is also preferred for R² and R²′ to represent a methyl group or a hydrogen atom, more preferably for each to represent a methyl group.

According to the invention, sophorolipids in which the acidic form and the lactone form are in a mixture are preferred, preferably approximately 20 to approximately 60% by weight of the sophorolipids being in the acidic form and the remainder of the sophorolipids being in the lactone form.

In particular, sophorolipids are preferred in which compounds of the above formulae (1) and (2) are present in a mixture, where R¹ and R^{1′} are a monounsaturated, linear hydrocarbon chain having 14 to 18 carbon atoms, more preferably 15 carbon atoms, R³ and R⁴ represent an acetyl group, R^{3′} and R^{4′} represent a hydrogen atom and R² and R2′ represent a methyl group, and approximately 20 to 60% by weight of the sophorolipids being in the acidic form.

Sophorolipids of this type can be obtained commercially, for example under the name Sopholiance S from Soliance. More precisely, the sophorolipid that can be obtained under the trade name Sopholiance S from Soliance is an approximately 60% by weight sophorolipid solution and is, for example, obtained by fermenting Candida bombicola on rapeseed oil methyl ester and glucose (INCI: Candida bombicola/glucose/methyl rapeseed ferment (and) water). Sopholiance S is a preferred sophorolipid according to the invention.

In Soliance S, approximately 20% by weight is present in the free acid form, in a mixture with the lactone form.

Mannosylerythritol lipids are glycolipids of the following general formula:

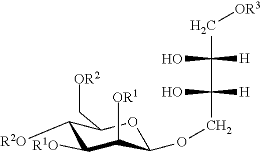

where R¹, independently of one another, represents fatty acid acyl groups having 4 to 24 carbon atoms, preferably 8 to 12 carbon atoms, R², independently of one another, represents a hydrogen atom or an acetyl group, and R³ represents a hydrogen atom or a fatty acid acyl group having 2 to 24 carbon atoms. A mannosylerythritol lipid that is suitable according to the invention can be obtained commercially under the name Ceramela-B (Toyobo) (INCI: Pseudozyma tsukubaensis/olive oil/glycerin/soy protein ferment).

The lipids and lipid derivatives substance group, to which in particular lipopeptides belong, are also included in the biosurfactants. In general, lipopeptides are synthesized non-ribosomally by the respective microorganisms, for example by Gram-positive bacteria, in particular of the genera Bacillus and Streptomyces, by Gram-negative bacteria, in particular of the genus Pseudomonas and by myxobacteria, as well as by filamentous plants. Normally, the peptide chains consist of two to forty amino acids, and can be linear, cyclic or branched. Unlike ribosomally synthesized peptide chains, lipopeptides often not only include proteinogenic L-amino acids as monomer structural elements, but also D-amino acids and carboxylic acids and/or all types of alpha-hydroxy carboxylic acids. The amino acids are usually L-α- or D-α-amino acids, although β-, γ- or δ-amino acids can also be present, which can likewise also be in a D- or L-configuration. The peptide chains can also include other chemical modifications; in particular they can be glycolyzed, hydrolyzed, N-methylated or N-formylated. Common structural elements are also thiazoline rings and/or oxazoline rings in various oxidation stages. A known lipopeptide biosurfactant is surfactin, which has the following structure and is generally used as an alkali salt or ammonium salt:

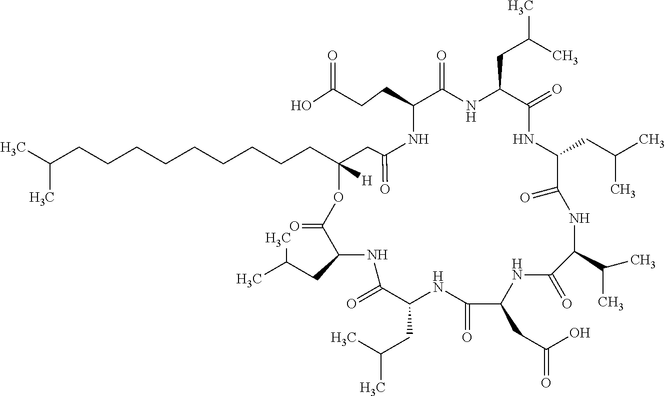 [SMILES: CC(C)CCCCCCCCC[C@@H]1CC(=O)N[C@@H](CCC(=O)O)C(=O)N[C@@H](CC(C)C)C(=O)N[C@H](CC(C)C)C(=O)N[C@@H](C(C)C)C(=O)N[C@@H](CC(=O)O)C(=O)N[C@H](CC(C)C)C(=O)N[C@@H](CC(C)C)C(=O)O1]

A surfactin that is suitable according to the invention can be obtained commercially from Kaneka.

The lipopeptides that can be preferably used as biosurfactants according to the invention also include fatty acyl glutamates. These have the following general formula:

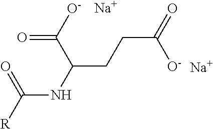

where R is a straight or branched alkyl chain having 5 to 21 carbon atoms, preferably 7 to 17 carbon atoms, more preferably 12 to 16 or 13 to 15 carbon atoms. Fatty acyl glutamates in the form of biosurfactants are generally provided in a mixture in which R has different chain lengths. The functional group R can also be hydroxylated, preferably monohydroxylated, in which case hydroxylation at the β-position is preferred. Fatty acyl glutamates in the form of biosurfactants can, for example, be obtained from Modular Genetics, Inc., USA.

The lipopeptides that can preferably be used according to the invention as biosurfactants also include fatty acyl glycinates. These have the following general formula:

RC(O)NHCH₂CO₂X,

where
R is a straight or branched alkyl chain having 5 to 21 carbon atoms, preferably 7 to 17 carbon atoms, more preferably 12 to 16 or 13 to 15 carbon atoms, and
X is a cation, preferably an alkali metal cation or an ammonium cation, more preferably a sodium or ammonium cation, or —H.

Fatty acyl glycinates in the form of biosurfactants can also be present in a mixture in which R can have different chain lengths.

Fatty acyl glycinates in the form of biosurfactants can be obtained, for example, from Modular Genetics, Inc., USA.

According to the invention, cosmetic cleansing agents that include the following biosurfactants are preferred: rhamnolipid(s) and/or sophorolipid(s) in the form of glycolipids, surfactin, fatty acyl glutamate and/or fatty acyl glycinate in the form of lipopeptides, and combinations thereof

The cosmetic cleansing agent preferably includes the biosurfactants in an amount of approximately 0.5 to 50% by weight, preferably approximately 0.5 to 20% by weight, more preferably approximately 1 to 10% by weight, even more preferably 1 to 5% by weight, based on the total weight of the cleansing agent. In the case of biosurfactant mixtures, the percentages relate to the total amount of biosurfactants included therein.

The cosmetic composition according to the invention includes, as a further essential component thereof, one or more anionic surfactants.

In principle, all anionic surface-active substances suitable for use on the human body are suitable as anionic surfactants. They are characterized by a water-solubilizing anionic group, such as a carboxylate, sulfate, sulfonate, or phosphate group, and a lipophilic alkyl group having approximately 8 to 30 C atoms. The molecule may also include glycol groups or polyglycolether groups, ester, ether and amide groups as well as hydroxyl groups. Examples of suitable anionic surfactants are, in each case in the form of the sodium, potassium, ammonium, and mono-, di-, and trialkanolammonium salts having 2 to 4 C atoms in the alkanol group,
ether carboxylic acids of the formula R—O—(CH₂—CH₂O)ₓ—CH₂—COOH, in which R is a linear alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
linear alkano sulfonates having 8 to 24 C atoms,
linear alpha-olefin sulfonates having 8 to 24 C atoms,
alpha-sulfo fatty acid methyl esters of fatty acids having 8 to 30 C atoms,
alkyl sulfates of formula R—OSO₃H, in which R is a preferably linear alkyl group having 8 to 30 C atoms,
alkyl polyglycol ether sulfates of the formula R—O—(CH₂—CH₂O)ₓ—OS₃OH, in which R is a preferably linear alkyl group having 8 to 30 C atoms and x=1 to 12,
mixtures of surface-active hydroxysulfonates,
sulfated hydroxyalkyl polyethylene glycol ethers and/or hydroxyalkylene propylene glycol ethers,
sulfonates of unsaturated fatty acids having 8 to 24 C atoms and 1 to 6 double bonds,
esters of tartaric acid and citric acid with alcohols that are products of the addition of approximately 2 to 15 molecules of ethylene oxide and/or propylene oxide to fatty alcohols having 8 to 22 C atoms, alkyl and/or alkenyl ether phosphates,
sulfated fatty acid alkylene glycol esters of formula RCO(AlkO)nSO₃M in which RCO— represents a linear or branched, aliphatic, saturated and/or unsaturated acyl group having 6 to 22 C atoms, Alk represents CH₂CH₂, CHCH₃CH₂ and/or CH₂CHCH₃, n represents numbers from 0.5 to 5 and M represents a cation, as are described in the,
amide ether carboxylic acids,
products of the condensation of C—C fatty alcohols with protein hydrolysates and/or amino acids and derivatives thereof, which are known to a person skilled in the art as protein fatty acid condensates, such as the Lamepon® types, the Gluadin® types, Hostapon® KCG, or the Amisoft® types.

Preferred anionic surfactants are selected from linear alkyl sulfates having 8 to 24 C atoms, ethylene oxide addition products thereof, and combinations thereof. Ethylene oxide addition products of the linear alkyl sulfates (alkyl ether sulfates) are particularly preferred. The anionic surfactant (b) is particularly preferably an alkyl ether sulfate having 10 to 18 C atoms, preferably 12 to 14 C atoms, and 1 to 6 ethylene oxide units, more preferably 2 to 4 ethylene oxide units, preferably a lauryl ether sulfate having 2 to 4 ethylene oxide units, even more preferably a sodium lauryl ether sulfate having two ethylene oxide groups.

The cosmetic cleansing agent according to the invention includes the anionic surfactant preferably in an amount of approximately 0.5 to 30% by weight, preferably approximately 1 to 20% by weight, more preferably approximately 2 to 15% by weight, even more preferably 3 to 10% by weight, based on the total weight of the cleansing agent. In the case of anionic surfactants mixtures, the percentages relate to the total amount of anionic surfactants included therein.

In preferred embodiments of the invention, aside from the biosurfactant and the anionic surfactant, the cosmetic cleansing agent does not include an additional surfactant or includes at least less than 0.5% by weight of an additional surfactant, preferably less than 0.2% by weight, more preferably less than 0.1% by weight, of an additional surfactant. An “additional surfactant” is intended to mean a non-ionic, cationic and amphoteric or zwitterionic surfactant.

In other embodiments, an additional surfactant can however be included, in particular a non-ionic surfactant, preferably in low amounts, however. An example of a non-ionic component having a surfactant effect is PEG-7 Glyceryl Cocoate (INCI).

The cosmetic cleansing agent of the present invention includes preferably 0.5% by weight or less of free fatty acid, more preferably no free fatty acid at all. A low content of fatty acids is advantageous in that the foaming behavior of the cosmetic cleansing agent is not inhibited.

The cosmetic cleansing agent of the present invention can be used in preservatives which are conventionally used in cosmetics. Preservatives used in cosmetics are generally used as broad-spectrum preservatives, and therefore also affect bacteria that are not specific to skin. As mentioned at the outset, these preservatives kill not only the undesired skin bacteria but also desired skin bacteria and thereby cause the biological balance of the skin to be disturbed. In the present invention, a prebiotic effect on skin is surprisingly observed despite the presence of conventional preservatives. Organic acids or salts thereof can be used as preservatives, for example, phenoxyethanol, methylparaben, ethylparaben, sodium benzoate, Na salicylate and combinations thereof. According to the invention, sodium benzoate, sodium salicylate or a combination thereof are preferably used as preservatives. Preferably, the cosmetic cleansing agent includes cosmetically acceptable preservatives in only low amounts, preferably in a total amount of 0.05 to 5% by weight or 0.1 to 3% by weight, 0.2 to 1% by weight or 0.2 to 0.7% by weight, based on the total weight of the cosmetic cleansing agent. If a plurality of preservatives are included, the percentages relate to the total amount of preservatives.

The cosmetic agent was found to have particularly good prebiotic effectiveness in the case of a combination of biosurfactants, in particular rhamnolipid and/or lipopeptide, with an alkyl ether sulfate, in particular lauryl ether sulfate having 2 to 4 ethylene oxide units as the anionic surfactant, and sodium benzoate and/or sodium salicylate as the preservative.

According to the invention, the cosmetic cleansing agent includes water as a cosmetic carrier. Other common carriers can be included in embodiments of the invention, but in terms of biodegradability and natural availability of the raw materials, it is preferred for the cosmetic cleansing agent according to the invention to only include water as the carrier.

According to the invention, the cosmetic cleansing agent can include additional conventional ingredients of cosmetic cleansing agents. Examples of conventional ingredients of this type are fragrances or perfumes, thickeners, opacifying agents, preservatives, pH regulators, for example citric acid, and/or care substances. However, the present invention is not restricted to these additional ingredients.

As thickeners, according to the invention, thickeners of plant origin are preferably included, such as polysaccharides like celluloses (cellulose itself and derivatives thereof), alginic acids (and the corresponding physiologically acceptable salts thereof, the alginates), agar agar (with the polysaccharide agarose present as the main constituent in agar agar), starch fractions and derivatives such as amylose, amylopectin and dextrin, karaya gum, gellan gum, carob gum, gum arabic, dextrane, guar gum and xanthan gum or combinations thereof. Guar gum, gellan gum and/or xanthan gum are preferable thickeners for the present invention.

Suitable cellulose derivatives are methyl celluloses, ethyl celluloses, hydroxyalkyl celluloses (such as hydroxyethyl cellulose), methyl hydroxyalkyl celluloses and carboxy methyl celluloses (INCI: cellulose gum) and the physiologically acceptable salts thereof

According to the invention, natural fragrances are preferred in particular as fragrances or perfumes that can be included in the cosmetic cleansing agent. If a fragrance is included, it is preferably included in an amount of 0.05 to 2% by weight, preferably 0.1 to 1.5% by weight, more preferably 0.2 to 1% by weight, and most preferably 0.5 to 1% by weight, in each case based on the total weight of the cleansing agent. If a plurality of fragrances are included, the percentages relate to the total amount of fragrances and/or perfumes.

As a care substance, the agent can, for example, include oil components, preferably natural oil components such as plant oils and plant extracts, but also monosaccharides or oligosaccharides and/or lipids. Aloe vera extracts or olive oil are mentioned as examples. Another suitable care substance is glycerol, which also acts as a moisture retention agent.

According to the invention, the cosmetic cleansing agent can be formulated as a body cleansing agent, a face cleansing agent or agent for cosmetically treating acne, such as a shampoo, shower gel, face gel or other known manufactured types of cosmetics.

Overview in tables:

Below is a list of the preferred cosmetic cleansing agents according to the invention. All the information is given in % by weight and relates to the active ingredient concentration.

| | Formula | Formula | Formula | Formula |
|---|---|---|---|---|
| | 1 | 2 | 3 | 4 |
| Biosurfactant | 0.5 to 50 | 0.5 to 20 | 1 to 10 | 1 to 5 |
| Anionic surfactant | 0.5 to 30 | 1 to 20 | 2 to 15 | 3 to 10 |
| Misc | ad 100 | ad 100 | ad 100 | ad 100 |
| | Formula | Formula | Formula | Formula |
| | 1a | 2a | 3a | 4a |
| Rhamnolipid, surfactin | 0.5 to 50 | 0.5 to 20 | 1 to 10 | 1 to 5 |
| and/or fatty acyl | | | | |
| glutamate | | | | |
| Alkyl ether sulfate | 0.5 to 30 | 1 to 20 | 2 to 15 | 3 to 10 |
| having 10 to 18 C atoms, | | | | |
| preferably 12 to 14 C | | | | |
| atoms, and 1 to 6 ethylene | | | | |
| oxide units, preferably | | | | |
| 2 to 4 ethylene oxide | | | | |
| units | | | | |
| Misc | ad 100 | ad 100 | ad 100 | ad 100 |
| | Formula | Formula | Formula | Formula |
| | 1b | 2b | 3b | 4b |
| Rhamnolipid, surfactin | 0.5 to 50 | 0.5 to 20 | 1 to 10 | 1 to 5 |
| and/or fatty acyl | | | | |
| glycinate | | | | |
| Alkyl ether sulfate | 0.5 to 30 | 1 to 20 | 2 to 15 | 3 to 10 |
| having 10 to 18 C atoms, | | | | |
| preferably 12 to 14 C | | | | |
| atoms, and 1 to 6 ethylene | | | | |
| oxide units, preferably | | | | |
| 2 to 4 ethylene oxide | | | | |
| units | | | | |
| Misc | ad 100 | ad 100 | ad 100 | ad 100 |
| | Formula | Formula | Formula | Formula |
| | 1c | 2c | 3c | 4c |
| Rhamnolipid, surfactin | 0.5 to 50 | 0.5 to 20 | 1 to 10 | 1 to 5 |
| and/or fatty acyl | | | | |
| glutamate | | | | |
| Sodium lauryl ether sulfate | 0.5 to 30 | 1 to 20 | 2 to 15 | 3 to 10 |
| having 2 to 4 ethylene | | | | |
| oxide units | | | | |
| Misc | ad 100 | ad 100 | ad 100 | ad 100 |
| | Formula | Formula | Formula | Formula |
| | 1d | 2d | 3d | 4d |
| Rhamnolipid, surfactin | 0.5 to 50 | 0.5 to 20 | 1 to 10 | 1 to 5 |
| and/or fatty acyl | | | | |
| glycinate | | | | |
| Sodium lauryl ether sulfate | 0.5 to 30 | 1 to 20 | 2 to 15 | 3 to 10 |
| having 2 to 4 ethylene | | | | |
| oxide units | | | | |
| Misc | ad 100 | ad 100 | ad 100 | ad 100 |
| | Formula | Formula | Formula | Formula |
| | 5 | 6 | 7 | 8 |
| Biosurfactant | 0.5 to 50 | 0.5 to 20 | 1 to 10 | 1 to 5 |
| Anionic surfactant | 0.5 to 30 | 1 to 20 | 2 to 15 | 3 to 10 |
| Preservative | 0.05 to 5 | 0.1 to 3 | 0.2 to 1 | 0.2 to 0.7 |
| Misc | ad 100 | ad 100 | ad 100 | ad 100 |
| | Formula | Formula | Formula | Formula |
| | 5a | 6a | 7a | 8a |
| Rhamnolipid, surfactin | 0.5 to 50 | 0.5 to 20 | 1 to 10 | 1 to 5 |
| and/or fatty acyl | | | | |
| glutamate | | | | |
| Alkyl ether sulfate | 0.5 to 30 | 1 to 20 | 2 to 15 | 3 to 10 |
| having 10 to 18 C atoms, | | | | |
| preferably 12 to 14 C | | | | |
| atoms, and 1 to 6 ethylene | | | | |
| oxide units, preferably | | | | |
| 2 to 4 ethylene oxide | | | | |
| units | | | | |
| Organic acids or salts | 0.05 to 5 | 0.1 to 3 | 0.2 to 1 | 0.2 to 0.7 |
| thereof, for example | | | | |
| phenoxyethanol, | | | | |
| methylparaben, | | | | |
| ethylparaben, sodium | | | | |
| benzoate, sodium salicylate | | | | |
| and combinations thereof | | | | |
| Misc | ad 100 | ad 100 | ad 100 | ad 100 |
| | Formula | Formula | Formula | Formula |
| | 5b | 6b | 7b | 8b |
| Rhamnolipid, surfactin | 0.5 to 50 | 0.5 to 20 | 1 to 10 | 1 to 5 |
| and/or fatty acyl | | | | |
| glycinate | | | | |
| Alkyl ether sulfate | 0.5 to 30 | 1 to 20 | 2 to 15 | 3 to 10 |
| having 10 to 18 C atoms, | | | | |
| preferably 12 to 14 C | | | | |
| atoms, and 1 to 6 ethylene | | | | |
| oxide units, preferably | | | | |
| 2 to 4 ethylene oxide | | | | |
| units | | | | |
| Organic acids or salts | 0.05 to 5 | 0.1 to 3 | 0.2 to 1 | 0.2 to 0.7 |
| thereof, for example | | | | |
| phenoxyethanol, | | | | |
| methylparaben, | | | | |
| ethylparaben, sodium | | | | |
| benzoate, sodium salicylate | | | | |
| and combinations thereof | | | | |
| Misc | ad 100 | ad 100 | ad 100 | ad 100 |
| | Formula | Formula | Formula | Formula |
| | 5c | 6c | 7c | 8c |
| Rhamnolipid, surfactin | 0.5 to 50 | 0.5 to 20 | 1 to 10 | 1 to 5 |
| and/or fatty acyl | | | | |
| glutamate | | | | |
| Sodium lauryl ether sulfate | 0.5 to 30 | 1 to 20 | 2 to 15 | 3 to 10 |
| having 2 to 4 ethylene | | | | |
| oxide units | | | | |
| Sodium benzoate and/or | 0.05 to 5 | 0.1 to 3 | 0.2 to 1 | 0.2 to 0.7 |
| sodium salicylate | | | | |
| Misc | ad 100 | ad 100 | ad 100 | ad 100 |
| | Formula | Formula | Formula | Formula |
| | 5d | 6d | 7d | 8d |
| Rhamnolipid, surfactin | 0.5 to 50 | 0.5 to 20 | 1 to 10 | 1 to 5 |
| and/or fatty acyl | | | | |
| glycinate | | | | |
| Sodium lauryl ether sulfate | 0.5 to 30 | 1 to 20 | 2 to 15 | 3 to 10 |
| having 2 to 4 ethylene | | | | |
| oxide units | | | | |
| Sodium benzoate and/or | 0.05 to 5 | 0.1 to 3 | 0.2 to 1 | 0.2 to 0.7 |
| sodium salicylate | | | | |
| Misc | ad 100 | ad 100 | ad 100 | ad 100 |

According to the invention, “Misc” is substantially understood to be water, optionally in combination with another cosmetic carrier, although the cosmetic carrier preferably only includes water. “Misc” may also optionally include other conventional ingredients of cosmetic cleansing agents, for example preservatives, care substances, pH regulators, such as acids, and/or fragrances.

In preferred embodiments, free fatty acids and other surfactants, in particular cationic and amphoteric surfactants, are not included in “Misc”.

### Examples

The following cosmetic cleansing agents set out in the tables were produced. The percentages are to be understood as percent by weight, based in each case on the total weight of the cleansing agent.

TABLE 1

| INCI or other name | Example 1 | Example 2 |
|---|---|---|
| FAEOS-Na C12-14 2 EO 70% | 11.00 | 11.00 |
| Water, demineralized | ad 100 | ad 100 |
| Sodium hydroxide 50% standard | 0.1078 | 0.1078 |
| Regular citric acid monohydrate | 0.16 | 0.16 |
| Water, demineralized | 54.9122 | 54.9122 |
| Rhamnolipid R-90 - biosurfactant | 0.84 | — |
| Lipopeptides | — | 0.77 |
| Nicotinamides | 0.01 | 0.01 |
| Terra-Pure Aloe Powder | 0.01 | 0.01 |
| Water, demineralized | 2.00 | 2.00 |
| Na benzoate | 0.40 | 0.40 |
| Sodium salicylate | 0.23 | 0.23 |
| Water, demineralized | 5.00 | 5.00 |
| Rhamnolipid R-90 - biosurfactant | 0.84 | 0.84 |
| Yoghurt Protein GBU | 0.05 | 0.05 |
| PEG-7 Glyceryl Cocoate | 0.30 | 0.30 |
| Hydra Vegetal Soap 992 perfume | 1.00 | 1.00 |
| Water, demineralized | 0.75 | 0.75 |
| Styrene acrylate copolymer OP 40% | 0.75 | 0.75 |
| Regular citric acid monohydrate | 0.15 | 0.15 |
| Sodium chloride fine-medium | 1.80 | 1.80 |
| Total | 100 | 100 |

Both compositions were found to have excellent overall cosmetic performance, including an excellent foaming behavior, washing behavior and feel on the skin/hair.

In vitro experiments also showed that the cosmetic cleansing agent of example 1 and 2 inhibited the growth of Propionibacterium acne and promoted the growth of Staphylococcus epidermis.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A cosmetic cleansing agent having prebiotic effectiveness, including one or more biosurfactants in combination with one or more anionic surfactants.

2. The cosmetic cleansing agent according to claim 1, which includes, based in each case on the total weight of the cosmetic cleansing agent:
(a) 1 to 20% by weight of the one or more biosurfactants and
(b) 1 to 10% by weight of the one or more anionic surfactants.

3. The cosmetic cleansing agent according to claim 1, which includes a glycolipid, a lipopeptide or a combination thereof as the biosurfactant (a).

4. The cosmetic cleansing agent according to claim 1, wherein the biosurfactant (a) is selected from the group consisting of rhamnolipids, sophorolipids, mannosylerythritol lipids, surfactins, fatty acyl glutamates, fatty acyl glycinates and combinations thereof.

5. The cosmetic cleansing agent according claim 1, wherein the anionic surfactant (b) is selected from the group consisting of linear alkane sulfates having 8 to 24 C atoms, ethylene oxide addition products thereof, and combinations thereof.

6. The cosmetic cleansing agent according to claim 1, which further includes one or more cosmetically acceptable preservatives in a total amount of 0.1 to 1% by weight, based on the total weight of the cosmetic cleansing agent.

7. The cosmetic cleansing agent according to claim 1, wherein the cosmetic cleansing agent does not include any free fatty acid.

8. The cosmetic cleansing agent according to claim 1, which is formulated as a body cleansing agent, face cleansing agent or agent for cosmetically treating acne.

9. A method of treating acne, which includes applying to skin the cleansing agent according to claim 1.

10. A method of treating the skin and imparting a prebiotic effect on the skin, which includes applying to the skin a prebiotically effective amount of the cleansing agent according to claim 1, wherein the biosurfactant are included in the agent as a prebiotically effective component.
